(19) United States
(12) Patent Application Publication
(10) Pub. No.: US 2014/0178918 A1
(43) Pub. Date: Jun. 26, 2014

(54) METHOD OF CHARACTERIZING PORES

(71) Applicant: Jin Young Chang, Trabuco Canyon (KR)

(72) Inventor: Jin Young Chang, Trabuco Canyon (KR)

(21) Appl. No.: 13/723,840

(22) Filed: Dec. 21, 2012

(51) Int. Cl. G01N 33/50

(52) U.S. Cl. CPC G01N 33/5008
USPC 435/29

(57) ABSTRACT

A method of characterizing one or more biomolecules in vivo is provided. More particularly, in order to monitor a plurality of biomolecules in a real time base, a plurality of labeling molecules that are not same and reactive to different wavelength can be used.

### BACKGROUND OF THE INVENTION

1. Field of the Invention

The present invention generally relates to a method of characterizing of measuring the size of connection part, for example, pores between two vesicles which have bio membrane thereof. More particularly, the invention relates to a method of measurement of pore size when two vesicles in vivo.

2. Description of the Related Art

It is very important to understand a vesicle pore in vivo since it is an important way to transfer or communicate between each cell. Therefore, many people have wanted to know the character of the pore, how large they are when they were generated. Many researchers have tried to measure it with various methods and I propose a complex which is composed of disulfide bond between simple chemicals and dyes. Thanks to use this molecular complex, it has been possible to measure a pore between two biomembranes. While this technique has been of great help, it still needs substantial improvement to provide a useful tool to study biological phenomenon. It is typical in biology that many different biomolecules are present and participate in a reaction to change a cellular event. Understanding the dynamics of participating molecular reaction of a pore and their dynamics would be of fundamental importance.

### SUMMARY OF INVENTION

### Detailed Description of the Preferred Embodiment

1. Disulfide bond

In chemistry, a disulfide bond (Br.E. disulphide bond) is a covalent bond, usually derived by the coupling of two thiol groups. The linkage is also called an SS-bond or disulfide bridge. The overall connectivity is therefore R—S—S—R. The terminology is widely used in biochemistry. In formal terms, the connection is a persulfide, in analogy to its congener, peroxide (R—O—O—R), but this terminology is obscure and is no longer used (except in reference to R—S—S—H or H—S—S—H compounds). The disulfide bond is strong, with a typical bond dissociation energy of 60 kcal/mole (251 kJ mol-1). However, being about 40% weaker than C—C and C—H bonds, the disulfide bond is often the “weak link” in many molecules. Furthermore, reflecting the polarizability of divalent sulfur, the S—S bond is susceptible to scission by polar reagents, both electrophiles and especially nucleophiles:

RS—SR+Nu−→RS−Nu+RS—

The disulfide bond is about 2.05 Å in length, about 0.5 Å longer than a C—C bond. Rotation about the S—S axis is subject to a low barrier. Disulfides show a distinct preference for dihedral angles approaching 90°. When the angle approaches 0° or 180°, then the disulfide is a significantly better oxidant.

Disulfides where the two R groups are the same are called symmetric, examples being diphenyl disulfide and dimethyl disulfide. When the two R groups are not identical, the compound is said to be an unsymmetric or mixed disulfide.

Although the hydrogenation of disulfides is usually not practical, the equilibrium constant for the reaction provides a measure of the standard redox potential for disulfides:

RSSR+H₂→2RSH

This value is about −250 mV vs NHE (pH=7). By comparison, the standard reduction potential for ferrodoxins is about −430 mV.

Formation of Disulfides

Disulfide bonds are usually formed from the oxidation of sulthydiyl (—SH) groups, especially in biological contexts. The transformation is depicted as follows:

2RSH→RS—SR+2H++2e−

A variety of oxidants promote this reaction including air and hydrogen peroxide. Such reactions are thought to proceed via sulfenic acid intermediates. In the laboratory, iodine in the presence of base is commonly employed to oxidize thiols to disulfides. Several metals, such as copper(II) and iron(III) complexes affect this reaction. [citation needed] Alternatively, disulfide bonds in proteins often formed by thiol-disulfide exchange:

RS—SR+R′SH R′S—SR+RSH

Such reactions are mediated by enzymes in some cases and in other cases are under equilibrium control, especially in the presence of a catalytic amount of base.

The alkylation of alkali metal di- and polysulfides gives disulfides. “Thiokol” polymers arise when sodium polysulfide is treated with an alkyl dihalide. In the converse reaction, carbanionic reagents react with elemental sulfur to afford mixtures of the thioether, disulfide, and higher polysulfides. These reactions are often unselective but can be optimized for specific applications.

Many specialized methods have been developed for forming disulfides, usually for applications in organic synthesis. Reagents that deliver the equivalent of “RS+” react with thiols to give asymmetrical disulfides:[3]

RSH+R′SNR″2→RS—SR′+HNR″2,

where R″2N phthalimido

### Scission of Disulfides

The most important reaction of disulfide bonds is their cleavage, which occurs via reduction. A variety of reductants can be used. In biochemistry, thiols such as mercaptoethanol (b-ME) or dithiothreitol (DTT) serve as reductants, the thiol reagents are used in excess to drive the equilibrium to the right:

RS—SR+HOCH₂CH₂SH HOCH₂CH₂S—SCH₂CH₂OH+2RSH

The reductant Tris(2-carboxyethyl)phosphine (TCEP) is useful, beside being odorless compared to b-ME and DTT, because it is selective, working at both alkaline and acidic conditions (unlike DTT), is more hydrophilic and more resistant to oxidation in air. Furthermore, it is often not needed to remove TCEP before modification of protein thiols.

In organic synthesis, hydride agents are typically employed for scission of disulfides, such as sodium borohydride. More aggressive, alkali metals will effect this reaction:

RS—SR+2Na→2NaSR

These reactions are often followed by protonation of the resulting metal thiolate:

NaSR+HCl→HSR+NaCl

Thiol-disulfide exchange is a chemical reaction in which a thiolate group —S— attacks a sulfur atom of a disulfide bond —S—S—. The original disulfide bond is broken, and its other sulfur atom (green atom in FIG. 1) is released as a new thiolate, carrying away the negative charge. Meanwhile, a new disulfide bond forms between the attacking thiolate and the original sulfur atom

Thiolates, not thiols, attack disulfide bonds. Hence, thiol-disulfide exchange is inhibited at low pH (typically, below 8) where the protonated thiol form is favored relative to the deprotonated thiolate form. (The pKa of a typical thiol group is roughly 8.3, but can vary due to its environment.)

Thiol-disulfide exchange is the principal reaction by which disulfide bonds are formed and rearranged in a protein. The rearrangement of disulfide bonds within a protein generally occurs via intra-protein thiol-disulfide exchange reactions; a thiolate group of a cysteine residue attacks one of the protein's own disulfide bonds. This process of disulfide rearrangement (known as disulfide shuffling) does not change the number of disulfide bonds within a protein, merely their location (i.e., which cysteines are bonded). Disulfide reshuffling is generally much faster than oxidation/reduction reactions, which change the number of disulfide bonds within a protein. The oxidation and reduction of protein disulfide bonds in vitro also generally occurs via thiol-disulfide exchange reactions. Typically, the thiolate of a redox reagent such as glutathione or dithiothreitol attacks the disulfide bond on a protein forming a mixed disulfide bond between the protein and the reagent. This mixed disulfide bond when attacked by another thiolate from the reagent, leaves the cysteine oxidised. In effect, the disulfide bond is transferred from the protein to the reagent in two steps, both thiol-disulfide exchange reactions.

The in vivo oxidation and reduction of protein disulfide bonds by thiol-disulfide exchange is facilitated by a protein called thioredoxin. This small protein, essential in all known organisms, contains two cysteine amino acid residues in a vicinal arrangement (i.e., next to each other), which allows it to form an internal disulfide bond, or disulfide bonds with other proteins. As such, it can be used as a repository of reduced or oxidized disulfide bond moieties.

2. General Characters of Dyes

According to another aspect of the invention, various dyes, which are often called as flurophore, that can be reactive to any type of laser can be used. A fluorophore (or fluorochrome, similarly to a chromophore) is a fluorescent chemical compound that can re-emit light upon light excitation. Fluorophores typically contain several combined aromatic groups, or plane or cyclic molecules with several π bonds. Fluorophores are sometimes used alone, as a tracer in fluids, as a dye for staining of certain structures, as a substrate of enzymes, or as a probe or indicator (when its fluorescence is affected by environment such as polarity, ions, . . . ). But more generally it is covalently bonded to a macromolecule, serving as a marker (or dye, or tag, or reporter) for affine or bioactive reagents (antibodies, peptides, nucleic acids). Fluorophores are notably used to stain tissues, cells, or materials in a variety of analytical methods, i.e. fluorescent imaging and spectroscopy. Fluorescein, by its amine reactive isothiocyanate derivative FITC, has been one of the most popularized fluorophores. From antibody labeling, the applications have spread to nucleic acids thanks to (FAM(Carboxyfluorescein), TET, . . . ). Other historically common fluorophores are derivatives of rhodamine (TRITC), coumarin, and cyanine. Newer generations of fluorophores, many of which are proprietary, often perform better (more photostable, brighter, and/or less pH-sensitive) than traditional dyes with comparable excitation and emission.

A. Fluorescence

The fluorophore absorbs light energy of a specific wavelength and re-emits light at a longer wavelength. The absorbed wavelengths, energy transfer efficiency, and time before emission depend on both the fluorophore structure and its chemical environment, as the molecule in its excited state interacts with surrounding molecules. Maximum wavelengths of absorption (≈ excitation) and emission (for example, Absorption/Emission=485 nm/517 nm) are the typical terms used to refer to a given fluorophore, but the whole spectrum may be important to consider. The excitation wavelength spectrum may be a very narrow or broader band, or it may be all beyond a cutoff level. The emission spectrum is usually sharper than the excitation spectrum, and it is of a longer wavelength and correspondingly lower energy. Excitation energies range from ultraviolet through the visible spectrum, and emission energies may continue from visible light into the near infrared region.

Main characteristics of fluorophores are:
Maximum excitation and emission wavelength (expressed in nanometers (nm)): corresponds to the peak in the excitation and emission spectra (usually one peak each),
Extinction Coefficient (or molar absorption, in Mol-1 cm-1): links the quantity of absorbed light, at a given wavelength, to the concentration of fluorophore in solution.
Quantum yield efficiency of the energy transferred from incident light to emitted fluorescence (=number of emitted photons per absorbed photons)
Lifetime (in picoseconds): duration of the excited state of a fluorophore before returning to its ground state. It refers to the time taken for a population of excited fluorophores to decay to 1/e (≈0.368) of the original amount.
Stokes shift: difference between the max excitation and max emission wavelengths.

These characteristics drive other properties, including the photobleaching or photoresistance (loss of fluorescence upon continuous light excitation). Other parameters should be considered, as the polarity of the fluorophore molecule, the fluorophore size and shape (i.e. for polarization fluorescence pattern), and other factors can change the behavior of fluorophores.

Fluorophores can also be used to quench the fluorescence of other fluorescent dyes (see article Quenching (fluorescence)) or to relay their fluorescence at even longer wavelength (see article FRET)

B. Size (Molecular Weight)

Most fluorophores are organic small molecules of 20-100 atoms (200-1000 Dalton—the molecular weight may be higher depending on grafted modifications, and conjugated molecules), but they are also much larger natural proteins: Green fluorescent protein (GFP) is 26 kDa and several phycobili proteins (PE, APC . . . ) are ≈240 kDa.

Fluorescence particles are not considered as fluorophores (quantum dot: 2-10 nm diameter, 100-100,000 atoms)

The size of the fluorophore might sterically hinder the tagged molecule, and affect the fluorescence polarity.

C. Families
Fluorescent proteins
GFP (green), YFP (yellow) and RFP (red) can be attached to other specific proteins to form a fusion protein, synthesized in cells after tranfection of a suitable plasmid carrier.
   Non-protein organic fluorophores belong to following major chemical families
Xanthene derivatives: fluorescein, rhodamine, Oregon green, eosin, and Texas red
Cyanine derivatives: cyanine, indocarbocyanine, oxacarbocyanine, thiacarbocyanine, and merocyanine
Naphthalene derivatives (dansyl and prodan derivatives)
Coumarin derivatives
Oxadiazole derivatives: pyridyloxazole, nitrobenzoxadiazole and benzoxadiazole
Pyrene derivatives: cascade blue etc.
Oxazine derivatives: Nile red, Nile blue, cresyl violet, oxazine 170 etc.
Acridine derivatives: proflavin, acridine orange, acridine yellow etc.
Arylmethine derivatives: auramine, crystal violet, malachite green
Tetrapyrrole derivatives: porphin, phtaloeyanine, bilirubin

These fluorophores fluoresce thanks to delocalized electrons which can jump a band and stabilize the energy absorbed. Benzene, one of the simplest aromatic hydrocarbons, for example, is excited at 254 nm and emits at 300 nm. This discrimates Fluorophores from quantum dots, which are fluorescent semiconductor nanoparticles.
They can be attached to protein to specific functional groups, such as
amino groups (Active ester, Carboxylate, Isothiocyanate, hydrazine)-carboxyl groups (carbodiimide)-thiol (maleimide, acetyl bromide)-azide (via click chemistry or non-specifically (glutaraldehyde)).

Additionally, various functional groups can be present to alter its properties, such as solubility, or confer special proprieties, such as boronic acid which binds to sugars or multiple carboxyl groups to bind to certain cations. When the dye contains an electron-donating and an electron-accepting group at opposite ends of the aromatic system, this dye will probably be sensitive to the environment's polarity (solvatochromic), hence called environment-sensitive. Often dyes are used inside cells, which are impermeable to charged molecules, as a result of this the carboxyl groups are converted into an ester, which is removed by esterases inside the cells, e.g., fura-2AM and fluorescein-diacetate.

The following dye families are trademark groups, and do not necessarily share structural similarities.
CF dye (Biotium)
BODIPY (Invitrogen)
Alexa Fluor (Invitrogen)
DyLight Fluor (Thermo Scientific, Pierce)
Atto and Tracy (Sigma Aldrich)
FluoProbes (Interchim)
DY and MegaStokes Dyes (Dyomics)
SulfoCy dyes (Cyandye)
Setau and Square Dyes (SETA BioMedicals)
Quasar and Cal Fluor dyes (Biosearch Technologies)
SureLight Dyes (APC, RPE, PerCP, Phycobilisomes)(Columbia Biosciences])
APC, APCXL, RPE, BPE (Phyto-Biotech)

3. Alexa Fluor Maleimides

Alexa Fluor dyes set new standards for fluorescent dyes and the bioconjugates prepared from them (The Alexa Fluor Dye Series—Note 1.1), Alexa Fluor dyes exhibit several unique features:

Strong absorption; with extinction coefficients greater than 65,000 cm-1M−1

Excellent photo stability, providing more time for observation and image capture than spectrally similar dyes allow

pH-insensitive fluorescence between pH 4 and pH 10

Superior fluorescence output per protein conjugate, surpassing that of other spectrally similar fluorophore-labeled protein, including fluorescein, tetramethylrhodamine and Texas Red conjugates, as well as Cy3 and Cy5 conjugates.

Note 1.1

| Derivative Abs* | Em* | Maleimide | Haloacetamide Bromomethyl | Halide | Cystine C or Thiosulfate T |
|---|---|---|---|---|---|
| Alexa Fluor 488 | 495 | 519 | A10254M | | |
| Alexa Fluor 532 | 532 | 553 | A10255 | | |
| Alexa Fluor 546 | 556 | 575 | A10258M | | |
| Alexa Fluor 555 | 555 | 565 | A20346 | | |
| Alexa Fluor 568 | 578 | 603 | A20341M | | |
| Alexa Fluor 594 | 590 | 617 | A10256M | | |
| Alexa Fluor 633 | 632 | 647 | A20342M | | |
| Alexa Fluor 647 | 650 | 665 | A20347 | | |
| Alexa Fluor 660 | 663 | 690 | A20343 | | |
| Alexa Fluor 680 | 679 | 702 | A20344 | | |
| Alexa Fluor 750 | 749 | 775 | A30459 | | |
| BODIPY FL 505 | 513 | B10250 | D6003 | | B20340C |
| T30453T | | | | | |
| BODIPY TMR | 542 | 574 | B30466 | | T30454T |
| BODIPY TR 589 | 617 | | | | T30455T |
| BODIPY 493/503 | 493 | 503 | | B2103 | |
| BODIPY 499/508 | 499 | 508 | D20350 | | |
| BODIPY 507/545 | 508 | 543 | D6004 | | |
| BODIPY 577/618 | 577 | 618 | D20351 | | |
| BODIPY 630/650 | 625 | 640 | | B22802 | T30456T |
| 4-Dimethylamino phenylazophenyl | 419 | NA | | D1521 | |
| Eosin 524 | 544 | E118 5 | | | |
| Fluorescein 494 | 518 | F150 5 | I30451 5 | | |
| I30452 6 B1355 5 | | | | | |
| Lucifer yellow 426 | 531 | | L1338 | | |
| NBD 478 | 541 | | I9† | | |
| D2004 | | | | | |
| F486 | | | | | |
| F6053‡ | | | | | |
| C20260 | | | | | |
| Oregon Green 488 | 496 | 524 | O6034 5 | O6010 M | |
| PyMPO | 415 | 570 | M6026 | | |
| QSY 7 | 560 | NA | Q10257 | | |
| QSY 9 | 562 | NA | Q30457 | | |
| QSY 35 | 475 | NA | | Q20348 | |
| Rhodamine Red | | 570 | 590 | R6029 M | |
| Sulfonerhodamine | | 555 | 580 | | B10621§ |
| Tetramethylrhodamine | | | 555 | 580 | T6027 5 |
| T6028 6 | | T6006 5 | | | |
| Texas Red 595 | | 615 | T6008M | | T6009M |

*Absorption (Abs) and emission (Em) maxima, in nm.
†Iodoacetate ester.
‡Like the NBD probes, ABD-F (F6053) is a benz-2-oxa-1,3-diazole, except that it is sulfonated (i.e., an SBD probe) instead of nitrated (i.e., an NBD probe); its reaction product with dimethylaminoethanethiol has Abs/Em maxima of 376/510 nm.
§Bifunctional crosslinker.
5 =5-Isomer.
6 = 6-Isomer.
M = Mixed isomers.
C = BODIPY FL L-cystine.
T = TS-Link fluorescent thiosulfate.
NA = Not applicable.

The Alexa Fluor maleimides are particularly useful for labeling thiol-containing proteins on the surface of live cells, where their polarity permits the sensitive detection of exposed thiols. In proteomics applications, Alexa Fluor protein conjugates can be electrophoretically separated and then detected without additional staining. As with their amine-reactive succinimidyl ester counterparts, Alexa Fluor 647 maleimide, Alexa Fluor 750 maleimide and other long-wavelength reactive dyes are frequently used to make conjugates for in vivo imaging applications. In experiments using Alexa Fluor 488 maleimide, immunodetection of labeled proteins can be accomplished using our anti-Alexa Fluor 488 antibody.

Photobleaching resistance of the green-fluorescent Alexa Fluor 488, Oregon Green 488 and fluorescein dyes, as determined by laser-scanning cytometry. EL4 cells were labeled with biotin-conjugated anti-CD44 antibody and detected by Alexa Fluor 488 (S11223, S32354), Oregon Green 488 (S6368) or fluorescein (S869). The cells were then fixed in 1% formaldehyde, washed and wet-mounted. After mounting, cells were scanned 10 times on a laser-scanning cytometer; laser power levels were 25 mW for the 488 nm spectral line of the argon-ion laser. Scan durations were approximately 5 minutes, and each repetition was started immediately after completion of the previous scan. Data are expressed as percentages derived from the mean fluorescence intensity (MFI) of each scan divided by the MFI of the first scan. Data contributed by Bill Telford, Experimental Transplantation and Immunology Branch, National Cancer Institute.

Photobleaching resistance of the red-fluorescent Alexa Fluor 647, Alexa Fluor 633, PBXL-3 and Cy5 dyes and the allophycocyanin fluorescent protein, as determined by laser-scanning cytometry. EL4 cells were labeled with biotin-conjugated anti-CD44 antibody and detected by Alexa Fluor 647 (S21374, S32357), Alexa Fluor 633 (S21375), PBXL-3, Cy5 or allophycocyanin (APC, S868) streptavidin. The cells were then fixed in 1% formaldehyde, washed and wet-mounted. After mounting, cells were scanned eight times on a laser-scanning cytometer; laser power levels were 18 mW for the 633 nm spectral line of the He—Ne laser. Scan durations were approximately 5 minutes, and each repetition was started immediately after completion of the previous scan. Data are expressed as percentages derived from the mean fluorescence intensity (MFI) of each scan divided by the MFI of the first scan. Data contributed by Bill Telford, Experimental Transplantation and Immunology Branch, National Cancer Institute.

4. Quantum Dot

A quantum dot is a portion of matter (e.g., semiconductor) whose excitons are confined in all three spatial dimensions. Consequently, such materials have electronic properties intermediate between those of bulk semiconductors and those of discrete molecules. They were discovered at the beginning of the 1980s by Alexei Ekimov in a glass matrix and by Louis E. Brus in colloidal solutions. The term “quantum dot” was coined by Mark Reed.

Researchers have studied quantum dots in transistors, solar cells, LEDs, and diode lasers. They have also investigated quantum dots as agents for medical imaging and hope to use them as qubits in quantum computing.

Stated simply, quantum dots are semiconductors whose electronic characteristics are closely related to the size and shape of the individual crystal. Generally, the smaller the size of the crystal, the larger the band gap, the greater the difference in energy between the highest valence band and the lowest conduction band becomes, therefore more energy is needed to excite the dot, and concurrently, more energy is released when the crystal returns to its resting state. For example, in fluorescent dye applications, this equates to higher frequencies of light emitted after excitation of the dot as the crystal size grows smaller, resulting in a color shift from red to blue in the light emitted. In addition to such tuning, a main advantage with quantum dots is that, because of the high level of control possible over the size of the crystals produced, it is possible to have very precise control over the conductive properties of the material. Quantum dots of different sizes can be assembled into a gradient multi-layer nanofilm.

In a preferred embodiment, a dynamics of the pore which was generated by proteins between two biomembranes can be studied. The simple chemicals can be any type of molecules including oligo- or polypeptide, nucleic acid, carbohydrate, and any derivatives as long as it contains —SH. As a bonded dye, any type of dyes molecules as listed in note 1.1 can also be used if you have appropriate light source to excite dyes. In studying dynamics of the pore size, each of the content molecules, which can be one or more in number, can be labeled with a different number of dye molecules. Consequently, the complex which is composed of the smallest number of dye is put inside of a vesicle. When two biomembranes meet and the pore is generated, the complex would be diffused from one vesicle to another vesicle. After trying to prepare the vesicle sample with different number of dye labeled, diffusion curve will show the drastic decreasing number of labeled dye complex. Since the chemicals are Circle shape, although they match the pore with any angle, we can expect the size of the pore. Accordingly, the technique according to the present application would allow significant improvement in understanding a molecular reaction as a whole in a real-time base.

In another preferred embodiment, the pore dynamics between the same kinds of molecules can be studied. For example, when two vesicles meet, the pore size can be plotted with time. Then we can understand in which step, how the pore size is getting bigger and maximum.

Molecules of interest comprise any biomolecules that may be present in vivo or in vitro. For example, mono-, oligo, poly-peptide and protein are included. Mono-, di-, and oligo-saccharide are included and any other forms of carbohydrate are included. A single molecule of lipid or any composite molecule comprising lipid is included. A single nucleic acid or poly-nucleic acid are also included. Additionally, any derivatives of the foregoing, e.g. post-transcriptionally modified nucleic acids, post-translationally modified proteins, lipids with carbohydrate functional groups, and others, are all included. Accordingly, it is noted that there is no limitation on types of biomolecules that can be a target of the methods/techniques that are disclosed in the present application.

The techniques and methods according to the invention can be applied to various topics of study. For example, the present techniques/methods can be used to measure the size of pores that participate a vesicle fusion of interest. If desired, in combination with measurement, monitoring diffusion dynamics of the lipid molecules can be performed in a real-time base. Further, we can compare the velocity from lipid molecules to the velocity of the pore growing. In addition, the techniques/methods can be of particularly powerful to explore the changes in the configuration of lipid bilayer which is the main component of cellular membranes. Given the dynamic nature of lipid bilayer, it is often difficult to reveal the molecular structure as well as quantifying the molecules present in the membrane. The cellular membrane is however often where many important reactions such as a signal transduction and immunological recognition would occur and understanding such a reaction would be necessary. With the techniques/methods disclosed herein, researchers can obtain data that may reflect the dynamics and structure in vivo. It is also worth noting that the techniques/methods can be useful to monitor a dynamics of the pore that participates a chain of reactions. Given the techniques/methods can be applicable in vivo, they can visualize the dynamic changes of the pore on a real-time base. Accordingly, for example, the techniques/methods are suitable to visualize a substantially whole chain of membrane fusion. Membrane fusions as discussed above is one of very important cellular reactions that may lead to molecular response in many biological events including neurological signal transduction as well as immunological response. Accordingly, it would be readily apparent to a person having ordinary skill in the art that the techniques/methods disclosed in the present application can be applicable to a variety of experimental and medical conditions without any limitations with no or substantially minor modifications. Therefore, any changes or modifications that can be done with routine procedures which are already known in the art should still be considered as being within the scope of the present invention.

A filed of application that can utilize the techniques/methods according to the present invention is also unlimited. Such a technique/method can be used, for example, a high-throughput screening of a pharmaceutical agent or drug that can lead to a certain cellular reaction. Therefore, in an experimental setting where one can monitor a configurational ro geological change of a interested molecule, the change can be monitored in vivo upon application of candidate agents or drugs. Therefore, for example, one can utilize such a technique/method to identify active novel compounds that can cause a certain neurological signal transduction or alternatively blocking the same. In addition, one can chase the molecular interaction between genetic materials such as RNA and DNA or between genetic materials and other transcription factors during gene expression or repression. Given that most of signal transduction reactions is led to modifications in genetic expression, it has been of particular importance to understand a chain of genetic regulation. With the techniques/methods according to the present invention, one can study (1) structural configuration of a single DNA, RNA or transcription factors, (2) interaction between two or more biomolecules during transcription, (3) geological variation of a certain molecule during gene expression, and (4) a substantially entire chain of gene expression. Specific fields of application and modification of the techniques/methods that may need to execute the desired application would be readily apparent to a person having ordinary skill in the art, and accordingly any application and modification of the techniques/methods disclosed in the present application should be considered as being within the scope of the invention.

For the purpose of instant illustration of a certain aspect of the invention without limiting the scope thereof, we present 9 Figures below and explain at least embodiments of the invention.

The pore is very important way to connect and to communicate between each cell. Essential chemicals, for example neurotransmitter or drug can be transferred through pores which are made by various kinds of proteins. Therefore, the interest of pores is getting important. There are two kinds of important factor for generation pores. The first one is how to make them and the second one is how large they are. In here I want to limit to pores which are made by SNARE proteins. Then how large pores are needed to transfer important chemicals? To understand this I propose one method. So far the verification of content mixing using DNA pairs has been well known so I want to go to next step. To investigate the size of pores before, many researchers have tried Quantum dot. However, since Quantum dot has different absorption with different diameter so we need various light sources to excite them. And it's not easy to put Quantum dot into the vesicle. It's not easy to distinguish the vesicle with Quantum dot from the vesicle without Quantum dot. Therefore my idea is using disulfide bond between commercial dyes and a simple chemicals. These chemicals can be small DNA molecules or other simple chemicals but it should be circle shape. The smallest one is labeling one site with dyes. If you want bigger one, you can make more systein substitution part and then label them with higher concentration of dyes. Then the general size of molecule grows and we can calculate the size of pores. Probably the size of one dye molecule can be calculated more exactly by molecular modeling and we can trust the calculated size of the pores. 1 and 2 show the experimental condition in detail. The green circle stands for a simple chemical and red ball means dyes. They bind into each other though disulfide bond like 2. Actual sample is not made like 1 and it is needed to prepare three kinds of samples respectively; a vesicle with the complex with just one dye, two dyes and three.

In this condition, the complex with one and two dyes can come out from the original vesicle, while the complex with three dyes cannot.

While various aspects and embodiments have been disclosed herein, other aspects and embodiments will be apparent to those skilled in the art. The various aspects and embodiments disclosed herein are for purposes of illustration and are not intended to be limiting, with the true scope and spirit of the present invention.

What is claimed is:

1. A method of characterize one or more biomolecules in vivo comprising:
associate a first labeling molecule to a first biomolecule;
associate a second labeling molecule to a second biomolecule, said first and second labeling molecules are not same and reactive to two separate wavelengths therefore they can be independently visualized,
collecting data indicative of signals from the first and second labeling molecules; and
computing the data.

2. The method according to claim 1, wherein the first and second labeling molecules are independently a nucleic acid probe, a protein probe, or a cellular functional probe.

3. The method according to claim 1, wherein the first and second labeling molecules are independently selected from the group consisting of CF dye, BODIPY, Alexa Fluor, DyLight Fluor, Atto and Tracy, FluoProbes, DY and MegaStokes Dyes, SulfoCy dyes, Setau and Square Dyes, Quasar and Cal Fluor dyes, Sure Light Dyes, APC, APCXL, RPE, and BPE.

4. The method according to claim 3, wherein the first and second labeling molecules are independently selected from the group consisting of CF dye, BODIPY, Alexa Fluor, DyLight Fluor, Atto and Tracy, and FluoProbes, DY.

5. The method according to claim 3, the first labeling molecule is selected from the group consisting of CF dye, BODIPY, and Alexa Fluor.

6. The method according to claim 3, the second labeling molecule is selected from the group consisting of DyLight Fluor, Atto and Tracy, and FluoProbes, DY.

7. The method according to claim 3, wherein said Sure Light Dyes comprise APC, RPE, PerCP, and Phycobilisomes.

8. The method according to claim 1, wherein said one or more biomolecules is selected from the group consisting of nucleic acid, peptide, carbohydrate, lipid and any derivatives thereof.

9. The method according to claim 1, wherein said any derivatives comprise a composite material comprising two or more selected from the group consisting of nucleic acid, peptide, carbohydrate, lipid.

10. The method according to claim 1, wherein the method is applied in vivo.

11. The method according to claim 1, wherein the method is applied in vitro.

12. The method according to claim 8, wherein the first biomolecule is a nucleic acid, and the second biomolecule is a protein.

13. The method according to claim 8, wherein the first biomolecule is a protein, and the second biomolecule is a carbohydrate.

14. The method according to claim 8, wherein the first biomolecule is a carbohydrate, and the second biomolecule is a lipid.

15. The method according to claim 8, wherein the first biomolecule is a protein, and the second biomolecule is a lipid.

16. The method according to claim 8, wherein the first biomolecule and the second biomolecule are a same kind of biomolecules.

17. The method according to claim 8, wherein the first biomolecule and the second biomolecule are two different kinds of biomolecules.

18. The method according to claim 1, wherein the method further comprises one or more additional labeling molecules.

19. A kit for characterizing one or more biomolecules in vivo comprising a first agent that is configured to label a first biomolecule and a second agent that is configured to label a second biomolecule wherein the first and second agents are not same and reactive to two separate wavelengths therefore they can independently visualize two different biomolecules.

20. The kit according to claim 1, wherein the first and second agents are independently selected from the group consisting of CF dye, BODIPY, Alexa Fluor, DyLight Fluor, Atto and Tracy, FluoProbes, DY and MegaStokes Dyes, SulfoCy dyes, Setau and Square Dyes, Quasar and Cal Fluor dyes, Sure Light Dyes, APC, APCXL, RPE, and BPE.
